(19) United States
(12) Patent Application Publication
(10) Pub. No.: US 2018/0168198 A1
(43) Pub. Date: Jun. 21, 2018

(54) NUTRITIONAL COMPOSITION

(71) Applicant: Rosemary Petralia, Merrimack, NH (US)

(72) Inventor: Rosemary Petralia, Merrimack, NH (US)

(21) Appl. No.: 15/844,951

(22) Filed: Dec. 18, 2017

Related U.S. Application Data
(63) Continuation of application No. 14/882,901, filed on Oct. 14, 2015, now abandoned.
Application No. 14/882,901 is a continuation of application No. 13/985,587, filed on Sep. 9, 2013, now abandoned.
Application No. 13/985,587 is a continuation of application No. 13/031,361, filed on Feb. 21, 2011, now abandoned.

(51) Int. Cl. A23L 2/52; A61K 36/886; A61K 35/08; A61K 33/14; A61K 31/352; A23L 33/16; A23L 33/00; A23L 27/00; A23L 33/15; A23L 33/105

(52) U.S. Cl. CPC A23L 2/52; A61K 36/886; A61K 35/08; A61K 33/14; A61K 31/352; A23V 2002/00; A23L 33/40; A23L 27/84; A23L 33/15; A23L 33/105; A23L 33/16

(57) ABSTRACT

A nutritional composition containing a plant food and a mineral food, where the plant food includes at least one of aloe, green algae, red algae, brown algae, blue-green algae, kelp, kombu, hijiki, nori, arame, sea palm, bladderwrack, wakame, dulse, carrageenan, fungi, and bacteria. The mineral food includes at least one of shilajit, humic acid, fulvic acid, bentonite clay, charcoal, diatomaceous earth, dolomite, fossilized remains, rocks, gems, sodium chloride, sea salt, and derivatives or combinations thereof. In one embodiment of the composition, the plant food is a juice of aloe vera and the mineral food is fulvic acid.

### RELATED APPLICATION(S)

This application is a continuation of U.S. application Ser. No. 14/882,901, filed on Oct. 14, 2015, which is a continuation of U.S. application Ser. No. 13/985,587, filed Sep. 9, 2013, now abandoned, which is a US National stage entry of International Application No. PCT/US2012/025924, which designated the United States and was filed on Feb. 21, 2012, published in English, which is a continuation of U.S. application Ser. No. 13/031,361, filed Feb. 21, 2011, now abandoned. The entire teachings of the above applications are incorporated herein by reference.

### BACKGROUND OF THE INVENTION

### 1. Technical Field

The present invention relates generally to chemical compositions and mixtures. More particularly, the present invention relates to a nutritional composition.

### 2. Background Art

Macro and micro-nutrients such as water, carbohydrates, proteins, lipids/fats, vitamins, minerals, trace minerals and electrolytes, enzymes, and accessory nutrients such phenolics and antioxidants, support a healthy life in humans and in animals. Studies show that adequate dietary macro- and micro-nutrient intake promotes health and wellbeing and slows the onset and progression of disease. However, most diets are deficient in these important nutrients due to their general lack of convenient availability in preferable forms.

Past nutritional compositions have consisted of artificial as well as natural ingredients. Additionally, past nutritional compositions often require flavor, color, and sugar/sweetener to increase visual and sensory appeal to consumers. Such additives make routine consumption increasingly questionable and undesirable.

### SUMMARY

Artificial ingredients are isolated or synthetic versions, or analogs, of natural nutrients and therefore lack the breadth, scope, and synergistic complexity of highly bioactive, bioavailable, and bioassimilable macro- and micro-nutrients found naturally in plant and mineral foods. These artificial compositions, as well as natural compositions, contain unsafe and undesirable preservatives, chemicals, synthetic additives, and contaminants like heavy metals. Past artificial and natural formulations are also unappealing in their appearance, taste, odor, and palatability. Many nutritive additives cannot be incorporated into other products without discernibly altering the substance's original or desired visual and sensory appeal.

The currently-available formulations lack the convenience of a single composition containing very broad-spectrum, 100 percent natural, organic, plant- and mineral-food-based macro- and micro-nutrients. Currently-available formulations also cannot be universally incorporated as a nutritive additive into many other various oral and parenteral delivery methods such as in waters, beverages, foods, feeds, condiments, confectioneries, herbal and dietary supplements, personal care and cosmetic products, and medicines.

Accordingly, a need exists in the art to provide a safe, convenient, and desirable form of a nutritional composition containing plant and mineral foods and that has multiple uses and forms.

Plant- and mineral-food-based nutrients provide the ideal sources of broad-spectrum nutrients in their most bioavailable form for the body's optimal utilization. These nutrients have been found scientifically safe, essential, and beneficial for human consumption and use. Therefore, a natural nutritional composition containing nutrients from most, or preferably all, of the major nutrient classes can safely and conveniently fill the nutritional gap in one's diet and potentially has positive implications on human and animal health and wellness when consumed regularly in various conveniently available forms.

It is an object of the present invention to provide a composition that is safe, effective, and beneficial for humans and other animals.

It is an object of the present invention to provide humans and animals with convenient oral and parenteral forms of nutrients.

It is an object of the present invention to provide a composition having improved nutrient content.

It is an object of the present invention to provide a composition containing bioactive micro- and macro-nutrients.

It is an object of the present invention to provide a composition having a variety of uses, including consumption and topical uses.

It is an object of the present invention to provide a nutritional composition containing plant and mineral foods.

The present invention achieves these and other objectives by providing composition having a plant food and a mineral food. The plant food is at least one of aloe, green algae, red algae, brown algae, blue-green algae, kelp, kombu, hijiki, nori, arame, sea palm, bladderwrack, wakame, dulse, carrageenan, fungi, and bacteria. The mineral food is one or more of shilajit, humic acid, fulvic acid, zeolite, bentonite clay, charcoal, diatomaceous earth, dolomite, fossilized remains, rocks, gems, sodium chloride, sea salt, and derivatives or combinations thereof.

In one embodiment of the present invention, the plant food is an extract of aloe vera, preferably aloe barbadensis miller.

In another embodiment, the aloe is substantially free of anthraquinones. In yet another embodiment, the aloe is at least 99% free of aloin and emodin.

In another embodiment of the present invention, the aloe contains less than 1 ppm aloin.

In another embodiment of the present invention, the plant food is substantially free of solids having a diameter greater than 5 μm. In another embodiment, the plant food is substantially free of solids having a diameter greater than 0.5 μm.

In another embodiment of the present invention, the composition has one or more of ascorbic acid, acetic acid, citric acid, malic acid, alcohol, honey, glycerin, salt, colloidal silver, potassium sorbate, sodium benzoate, vitamin E, astaxanthin, grape seed extract, and rosemary extract.

In another embodiment of the present invention, the composition has at least 1000 mg of mucopolysaccharides per liter.

In another embodiment of the present invention, the mineral food is fulvic acid, preferably a fermentation- or carbohydrate-derived fulvic acid liquid.

In another embodiment of the present invention, the fulvic acid contains at least 1 part dibezo-alpha-pyrones per 20 parts fulvic acid.

In another embodiment of the present invention, the composition is an aqueous solution.

In another embodiment of the present invention, the aqueous solution is substantially clear, substantially colorless, substantially odorless, and/or substantially flavorless.

In another embodiment of the present invention, the composition has a fulvic acid complex of least one of Fe³⁺, Al³⁺, and Cu²⁺.

In another embodiment of the present invention, the composition has a fulvic acid mineral complex.

In another embodiment of the present invention, the composition has a volume-to-volume ratio of plant food to mineral food that is between 2:1 and 1:2.

In another embodiment of the invention, the composition contains from 20 to 50 grams of fungi and/or bacteria per liter of plant food.

In another embodiment of the invention, the composition has one or more of alcohols, alkaloids, auxins, carbohydrates, proteins, amino acids, enzymes, hormones, caroteinoids, glycosides, lignans, lipids, microorganisms, minerals, zeolites, nucleic compounds, pharmaceutical drugs, “smart” drugs, phenolic compounds, quinones, vitamins, gases, herbs, foods, genetically modified organisms, microorganisms, nucleic compounds, organic acids, inorganic acids, cosmeceuticals, bioceuticals, dermaceuticals, nanoceuticals, quantum-altered substances, nanoparticles, and lyposomes. The ratio of the plant food(s) to the substance(s) is 10:1 or less.

In an embodiment for a method of making a nutritional composition, the method includes the step of providing at least one plant food one or more plant foods from aloe, green algae, red algae, brown algae, blue-green algae, kelp, kombu, hijiki, nori, arame, sea palm, bladderwrack, wakame, dulse, carrageenan, fungi, and bacteria. The method also includes the step of providing one or more mineral foods from shilajit, humic acid, fulvic acid, bentonite clay, charcoal, diatomaceous earth, dolomite, fossilized remains, rocks, gems, sodium chloride, sea salt, and derivatives or combinations thereof. The method also includes the step of mixing the plant food(s) with the mineral food(s) where the ratio of the plant food(s) to the mineral food(s) is from about 1:3 to about 4:1. In one embodiment, the mixing step is performed in water.

In another embodiment, the method also includes the step of removing at least one of aloein, aloe-emodin, anthraquinones, aloe latex, and particles having a diameter greater than 5 microns.

In another embodiment, the method also includes the step of adding one or more of acetic acid, citric acid, ascorbic acid, malic acid, alcohol, honey, glycerin, salt, colloidal silver, potassium sorbate, sodium benzoate, vitamin E, astaxanthin, grape seed extract, and rosemary extract.

In another embodiment, the method includes the step of adding at least one or more of alcohols, alkaloids, auxins, carbohydrates, proteins, amino acids, enzymes, hormones, caroteinoids, glycosides, lignans, lipids, microorganisms, minerals, zeolites, nucleic compounds, pharmaceutical drugs, “smart” drugs, phenolic compounds, quinones, vitamins, gases, herbs, foods, genetically modified organisms, microorganisms, nucleic compounds, organic acids, inorganic acids, cosmeceuticals, bioceuticals, dermaceuticals, nanoceuticals, quantum-altered substances, nanoparticles, and lyposomes. The ratio of the at least one plant food to the at least one ingredient is 10:1 or less.

### BRIEF DESCRIPTION OF THE FIGURES

FIG. 1 illustrates various embodiments of a method of making a nutritional composition of the present invention.

### DETAILED DESCRIPTION OF THE PREFERRED EMBODIMENT

The composition of the present invention contains two main components, namely a plant food and a mineral food. In one embodiment, the plant food is aloe and the mineral food is fulvic acid. In other embodiments, the mineral food is either humate, or a derivative thereof, such as humic-fulvic complex or synthetic analogs of humate.

Plant foods include aloe, algae, blue-green algae, fruit, fungi (edible), grains, legumes, nuts, plant oils, pollen, seeds, vegetables, sea vegetables, herbs, and processed plant foods thereof. Plant foods are derived from plant sources and are commonly consumed either for their taste or therapeutic benefits. Plant foods like aloe are a source of macro- and micro-nutrients vital to support life.

Mineral foods include humic substances, such as humic acid, fulvic acid and humic-fulvic complexes. Mineral foods also include minerals such as inorganic salts, organic salts, and amino acid chelates. Examples of minerals are zeolites, bentonite clay, charcoal, diatomaceous earth, salts, and sea minerals.

Plant Foods

Aloe is a botanical in a genus containing about four hundred species of flowering succulent perennial plants belonging to the lily family (Liliaceae). The most common and well known of these is aloe vera (L.) Brum F. (aloe barbadensis miller).

The aloe plant is an extraordinary plant containing vitamins, minerals, nutrients, enzymes, anti-inflammatory fatty acids, mannose, galactose and glycoprotein. For centuries, aloe has been used as a natural treatment for many health conditions including digestive conditions (colitis, Crohn's disease, and digestive tract impairments), circulatory, inflammatory and lymphatic conditions, auto-immune disorders, Lupus, fibromyalgia, chronic fatigue, rheumatoid arthritis, cancer, multiple sclerosis and many diseases. While aloe vera will be used in describing the properties and constituents, this information also applies to aloe.

Aloe vera is available in two basic raw forms, namely a gel and a liquid. The gel and the liquid both refer to the juice, gel or liquid that is extracted from the aloe vera leaf. Depending on the application, the gel may be extracted from either the whole leaf or from the inner leaf. This raw product has many uses, both internal and external. As extracted from the inner leaf, 100 ml of aloe vera juice contains 99.5 ml of water and 0.5 g (0.5 ml) of total solids. The aloe vera juice's total solids contain 3.65% methanol-precipitated (polysaccharide) solids (“MeOHPS”). The aloe vera juice also contains 1,800 mg/L of polysaccharides in the MeOHPS, 0.05% immune modulatory molecules (very large chain polysaccharides such as acemannan, described below) having a molecular weight of 2E6 Daltons, 7.02% inflammation modulatory molecules (small chain mucilaginous polysaccharides or mucopolysaccharides, described below) with a molecular weight of 6.6E4 Daltons, and 48.2 ppm laxative molecules (anthraquinones and aloe latex, described below).

The gel and liquid can both be concentrated by removing water. For example, the gel may be desiccated into a powder. The powder can be added back into to the gel or liquid to increase product concentration and potency. Aloe vera concentrate is aloe vera gel/juice from which natural water has been mechanically removed to a desired level. Reconstituted aloe vera gel/juice is freeze-dried aloe vera gel/juice to which an appropriate amount of water is added to achieve a concentration that is equivalent to 100% aloe vera gel/juice as it is extracted directly from the leaf. In reconstituted aloe vera gel/juice, the aloe vera may be freeze dried with or without a matrix.

Aloe Vera Constituents

The main components and constituents of aloe vera can be divided into groups that include amino acids, anthraquinones, carbohydrates, mucopolysaccharides, enzymes, hormones, glucoproteins, lectin, lignin, lipids and fatty acids, sterols, minerals, organic compounds, saponins, vitamins, water, and other constituents.

Amino Acids.

Amino acids provide the basic building blocks of proteins in the production of connective tissue such as cells, muscles, collagen, etc. The body needs 22 amino acids. Aloe provides many or all 22 of these required amino acids including the 8 essential amino acids which the body cannot synthesize. These amino acids include: isoleucine, leucine, lysine, methionine, phenylalanine, threonine, valine, tryptophan, as well as alanine, arginine, asparagine, cysteine, glutamic acid, glycine, histidine, ornothine, proline, hydroxyproline, serine, tyrosine, glutamine, and aspartic acid.

Anthraquinones (Quinones) or Phenolics.

In relatively low concentrations, anthraquinones (Quinones) or Phenolics provide analgesic, antibacterial, antifungal, antiviral, and antitubercular activity. Typically, laxative concentrations of less than 0.1 ppm are used without a purgative effect. In contrast, laxative concentrations of up to 50 ppm or more are used as potent laxatives. Anthraquinones in aloe vera are considered to provide safe and effective purgative effect at concentrations below 5 ppm. Twelve phenolic compounds are found exclusively in the plant sap. In small quantities, when they do not exert their purgative effect, phenolic compounds aid absorption in the gastrointestinal tract, have antimicrobial properties, and relieve pain. In some ingestible products, anthraquinones are removed to avoid side effects, such as producing abdominal pain or cramping, diarrhea, electrolyte imbalances, and other health-related conditions. Anthraquinones, however, are beneficial in small quantities. For example, aloin and aloe-emodin both relieve pain; barbaloin has an anti-thromboxane and an anti-prostaglanid effect. Emodin and barbaloin also function as antibacterials and antivirals and can be broken down into calculates for increased analgesia.

Twelve phenolic compounds comprise the anthraquinone complex, including aloe-emodin, aloesin/aloetic acid, aloin, anthracine, anthranol, barbaloin, chrysophanol/chrysophanic acid, aloe-emodin, ethereal oil, ester of cinnamonic acid, isobarbaloin, and resistannol. In general, phenolic compounds are pharmaceutically active, mildly antiseptic, antibiotic, and anti-inflammatory.

Carbohydrates.

Aloe contains monosaccharides and polysaccharides. Monosaccharides contain simple sugars which include glucose and fructose that have an anti-inflammatory effect. Polysaccharides are more complex, long-chain sugars involving glucose and mannose. Polysaccharides also involve glucomannans and polymannose with anti-viral and immune-modulating activity. The entire group of small- to very-large-chain polysaccharides is collectively referred to as glucomannans. Unlike other sugars, glucomannan polysaccharide sugars are ingested from the stomach without being broken down and appear in the blood stream in exactly the same form. This process is known as pinocytosis. Once in the blood stream, they exert their healing and immuno-regulating effect. Some of these polysaccharides are not absorbed, but stick to certain cells lining the gut and form a barrier preventing absorption of unwanted material. This mechanism helps to prevent intestinal permeability or “leaky gut” syndrome.

Acemannan is a polysaccharide known for its ability to restore and boost the immune system by stimulating macrophage production and T-lymphocyte activity by up to 50%. Acemannan produces immune agents interferon and interleukin, which help destroy viruses, bacteria, and tumor cells. Acemannan improves cellular metabolism by normalizing cellular function and regulating the flow of nutrients and wastes in and out of the cells. It is able to destroy parasites and fungus. Additional sugars found in aloe include arabinose, galactose, xylose, as well as the polysaccharides, mannose-6-phosphate, cellulose, mannose, (Hexo)uronic acid, glucuronic acid, galacturonic acid, aldonentose, L-rhamnose, and peptic acid.

Polysaccharides are often referred to as mucopolysaccharides (MPS), a high molecular weight complex carbohydrate that interfaces directly with the cells of the immune system to balance and bolster their biochemical activity. The higher molecular weight MPS are known as long-chain, high molecular weight carbohydrates.

Aloe vera contains beneficial mucilaginous polysaccharides (MPS). Aloe MPS molecules contain a full range of aloe vera glyconutrients, from the lightweight short chain molecules to the heavyweight long chain molecules. The varying sizes of the MPS molecules determine the healing properties.

Small size MPS have approximately 50 to 600 molecules and a mass below 50,000 Daltons. These smaller molecules reduce inflammation related to diseases such as ulcerative colitis, arthritis, and gastric reflux. These molecules may also help reduce blood sugar with both type I and II diabetes.

Medium size MPS have up to 1,500 molecules and a mass between 50,000 and 150,000 Daltons. While vitamins and minerals can only function outside the cells, mucopolysaccharides are very effective intracellular antioxidants and free radical scavengers. This quality may help with arteriosclerosis, heart disease and Parkinson's disease.

Large size MPS have up to 5,000 molecules and a mass between 150,000 and 1 million Daltons. These polysaccharides may have a direct anti-bacterial and anti-viral effect, which is important to combat new infectious diseases and more virulent strains of older diseases.

Very large size MPS have up to 9,000 molecules and a mass of more than 1 million Daltons. These very large polysaccharides are immune modulating, which may be helpful to different immune system disorders. It is also these very large molecules (TNF blockers) that may have an inhibitive effect on tumor necrosis factor, making aloe a promising prophylactic candidate.

Mucopolysaccharides (MPS) Molecular Weights.

Molecular weights of MPS molecules that occur in aloe vera have approximate upper and lower ranges as shown in Table 1.

TABLE 1

| Molecular Weight (Daltons) | Percentage |
|---|---|
| 0-66,000 | 0-72.7% |
| 66,000-150,000 | 3.1-24% |
| 150,000-200,000 | 0.4-5.9% |
| 200,000-500,000 | 0.1-14% |
| 500,000-1,000,000 | 0.7-79.4% |
| 1,000,000-2,000,000 | 0.2-2.7% |
| 2,000,000+ | 0-7.7% |

As shown in Table 1, 0.0%-72.7% of MPS have molecular weight from 12,500 to 66,000 Daltons; 3.1%-24% have molecular weight from 66,000 to 150,000 Daltons; 0.4% to 5.9% have molecular weight from 150,000 to 200,000 Daltons; 0.1%-14% have molecular weight from 200,000-500,000 Daltons; 0.7%-79.4% have molecular weight from 5,000,000 to 1,000,000 Daltons; 0.2%-2.7% have molecular weight from 1,000,000 to 2,000,000 Daltons; and 0-7.7% have molecular weights above 2,000,000 Daltons.

Enzymes.

Enzymes assist in the digestion of food, sugars, and fats, aiding digestive processes and enhancing nutrient absorption. They are essential to chemical reactions of vitamins, minerals, and hormones to take place. Several different types of these biochemical catalysts when taken orally aid digestion by breaking down fat and sugars. In particular, bradykinase, helps to reduce excessive inflammation when applied to the skin topically and therefore reduces pain, whereas others help digest any dead tissues in wounds. Lipases and proteases, or proteolytic enzymes, are present, which break down foods and aid digestion. In addition to bradykinase, other enzymes present in aloe vera include aliinase, alkaline phosphatase, alpha-amalyse, amylase, carboxypeptidase, catalase, cellulose, creatine phospkinase, gamma transaminase, glutathione peroxides, proteolytiase, glyoxalase I, and glyoxalase II, lactic dehydrogenase, lipase, oxidase, peroxidase, and SGO transaminase, SGP transaminase, superoxide dismutase. Since enzymes and amino acids are highly interactive and often assume personalities of one another and since the presence of nearly complete amino acids complex signals the potential for amplification of a number of other yet-to-be-identified elements, the potential for another twenty or thirty members of the enzyme family is very likely.

Hormones.

Aloe vera contains two hormones that provide wound healing and anti-inflammatory activity: Auxins and Gibberellins. Gibberellin acts as a growth hormone stimulating the growth of new cells.

Glucoproteins.

Aloe contains the glucoproteins alexin a. and alexin b. These substances possess hemagglutinating (blood coagulating) and cytoagglutinating (cell fixating) properties and serum protein binding properties. They are effective in acting against cancer, inflammation, burns and skin disease as well as act as anti-bacterials, controlling bacterial proliferation.

Lectin.

Aloectin a. is an anti-tumor agent and prevents edema and arthritis.

Lignin.

By itself, lignin is an inert, cellulose-based substance. When included in topical preparations it endows aloe vera with a singular penetrative effect, carrying other elements with it so the other ingredients are absorbed into the skin. It also acts as a carrier for other components of aloe vera. Lignin takes the soothing properties of aloe vera into the cells where it increases blood circulation to the area and flushes the necrotic tissue away. It is also keratolytic, meaning it destroys and softens hard skin.

Lipids/Fatty Acids.

Lipids in combination with proteins and carbohydrates constitute the principal structural components of living cells and carry fat soluble vitamins as well as supplies fatty acids and energy to the skin and body. The lipid category is comprised of isoprenoids, alkanes, n alkyl alcohols, gamma-linolenic, fatty acids and their esters, gamma-linolenic and linoleic as well as arachadonic acid, lecithin, and triglycerides. Fatty acids are responsible for the synthesis of prostaglandins and membrane structure.

Sterols.

Aloe vera contains four plant steroids that are important anti-inflammatory and antibacterial agents include cholesterol, campesterol, beta-sitosterol and lupeol. Lupeol also possesses antiseptic and analgesic properties.

Minerals.

Essential for good health and known to work in certain combination with each other as well as with trace minerals, vitamins, and amino acids. The minerals in aloe vera include calcium, sodium, potassium, manganese, magnesium, chlorine, copper, geranium, iron, silicon, sulfur, zinc, chromium, and selenium (an antioxidant). Although minerals and trace elements are only needed in very small quantities, they are essential for the proper functioning of various enzyme systems in different metabolic pathways.

Organic Compounds.

In addition to chrysophanic acid, aloe vera contains cinnamonic acid and salicylic acid. Cinnamonic acid is an anti-microbial and anti-helminitic compound. Salicylic acid, which, along with lactates, possesses analgesic, anti-inflammatory and antimicrobial properties. Aloe vera also contains monosulfonic acid, succinic acid, uric acid, traumatic acid (a wound-healing hormone) hyaluronic acid (a subcutaneous cementing substance), and urea-nitrogen (a pain-killing agent).

Saponins.

These glycosides are the soapy substances that form about three percent (3%) of aloe vera. Saponins are capable of cleansing, they have antiseptic properties, and they have effective specifying agents to provide natural surfactant properties. Saponins act powerfully as antimicrobials against bacteria, viruses, fungi and yeasts.

Vitamins.

Aloe vera is rich in all vitamins except vitamin D. Aloe vera is especially rich in vitamins A (free-radical-reducing antioxidant), C, E, and beta-carotene. Aloe even contains trace amounts of vitamin B12, making it one of the very few plant sources of this vitamin. Vitamin A and beta-carotene contribute to the integrity of the epithelium. Vitamin C is essential to collagen formation, tissue repair, and wound healing as well as deactivates free oxygen radicals responsible for inflammation. Vitamin B2 (riboflavin) is important to mucous membrane integrity. Folic acid is used for red blood cell maturation and is important for vegetarians and vegans. Additional vitamins found in aloe vera include the B-complex vitamins including B, B3, BS, B6, and choline. The B vitamins and choline are involved in amino acid metabolism whereas vitamin B12 is required to produce oxygen-carrying red blood cells.

Water.

Aloe vera's main constituent is water. Aloe vera generally contains 96,000 milligrams of water per 100 grams (i.e. 96% water by mass). Water is the “universal solvent” and has a hydrating and homeostatic effect on cells.

Other Constituents.

In addition to phenolic glycosides including dihydrocoumarins, aloe also contains resin, rhabarberone, and triterpene (a phytonutrient that lowers blood sugar), and prostanoids, a subclass or eicosanoids consisting of prostaglandins (mediators of inflammatory and anaphylactic reactions) thromboxanes (mediators of vasoconstriction), and prostacyclins (active in the resolution phase of inflammation).

Aloe Vera Biological Activity

Aloe vera applications were carefully documented from a variety of professional reports and measured against a consistent set of criteria that form a basic standard for all drugs, therapies, and curative treatments. These criteria are described below.

Healing Acceleration.

Aloe quickly and effectively heals damaged (or necrotic) tissue by stimulating the activity of fibroblasts which are extremely important in wound healing, because these fibers create a mesh or network over which the new skin cells advance to close the wound.

Reduction of Swelling.

Swelling is often the body's way of trying to heal itself.

Anti-Inflammatory Qualities.

Inflammation is injured tissue's alarm system. Aloe vera is unequalled in its anti-inflammatory properties.

Enzymatic Debridement.

Aloe can rapidly debride or break down the damaged and diseased tissue.

Pain Relief.

Aloe is a potent analgesic.

Antiseptic.

Aloe vera is highly antiseptic and may be used for cleaning wounds or damaged tissue without concomitant negative effect to tissue such as scarring.

Antipruritic.

Aloe is relieves itching and therefore speeds healing caused by scratching, particularly in the case of wounds, burns or topical diseases.

Penetration.

In order for a medication to be effective it must penetrate tissue. Penetration is equally important to topical and deep healing.

Bactericidal.

One of the most important healing aspects is the ability to efficaciously kill bacteria. Aloe vera has proven to be consistently effective against multiple drug resistant microbes when nothing else worked.

Virucidal.

Viruses are resistant to many common modalities, including antibiotics. Aloe vera has successfully killed viruses in many applications.

Fungicidal.

Fungi, such as Candida albicans tends to proliferate at mucous membranes and can go unnoticed due to their often low antigen profile. Candida infections commonly situate in the reproductive and digestive systems. Aloe vera has been proven in lab tests to be one-hundred percent (100%) effective against Candida albicans in solutions containing as little as fifty percent (50%) aloe. Aloe has similar efficacy against other fungi.

Trichomacidal.

Trichoma stems from a single cell protozoan and feeds on reproductive organ infections. Aloe vera is highly effective against this organism.

Antiedemic.

Edema is the accumulation of abnormal fluid. Aloe vera invaluably assists in draining and suppressing fluid build-up.

Hemostatic.

Hemostasis is the process by which blood clots, an essential would-healing process. Aloe vera has been found to be highly effective as a blood coagulant as well as a blood anti-coagulant. This unique property makes Aloe vera thermostatic.

Moisturizing.

Many medications can be drying and damage tissue with long-term use. Aloe vera contains natural humectants that act like moisturizers. This is particularly useful for esthetic and cosmetic applications.

Toxicity.

Aloe vera consistently lacks side effects and contraindications in virtually all treated patients. This reinforces the belief that aloe vera is virtually non-toxic at any level of use.

Biological Vehicle.

Aloe can solubilize water soluble compounds as well as lipid soluble substances. It can hydrate the skin and mucous membranes to aid a range of materials in penetrating the skin. Aloe's biological activity can add and even synergize with many agents in producing therapeutic effects, making aloe a biological vehicle. This may adequately explain its synergistic relationship with humic and fulvic acid's many agents.

Digestive Aid.

A healthy digestive tract ensures that nutrients from the food we eat are absorbed into the bloodstream. Aloe vera enables the bowel to absorb these nutrients more efficiently, especially protein.

In one embodiment, the composition contains about fifteen to about thirty 15-30 ml of food-grade, fractionally-distilled, one-hundred percent (100%) aloe vera juice extract from the leaves of aloe barbadensis miller. When mixed with about one to two liters of water, the resulting solution is substantially indistinguishable from water in clarity, color, odor, and taste. That is, the solution is clear, colorless, odorless, and tastes like spring water. Further, because the aloe vera juice extract is fractionally distilled, the solution is free of preservatives, additives, latex, anthraquinones, and aloin. The solution contains no binders, fillers, excipients, dyes, or foreign substances or contaminants. In an alternative embodiment, the aloe vera juice is processed to meet medicinal purity and quality standard by using micron filtration between one half (0.5) and five (5) microns.

One processing method removes the bitter sap and latex fractions of aloe vera while retaining vitamins, oils, and minerals. An aloe vera mash is mixed with food-grade ethanol and water. Ethanol dissolves most of the components in aloe vera that are not water soluble. The mixture is allowed to settle, it is filtered, and the resulting azeotropic liquid is distilled. During distillation, most of the desired ingredients are dissolved and transported to a collection vessel. The collection vessel is placed uncovered on low heat to evaporate the ethanol, resulting in an aqueous mixture containing active aloe ingredients. Alternately, the liquid mixture is fractionally distilled to remove the ethanol, leaving an aqueous solution with active aloe ingredients.

The amount of the aloe vera used in the composition may vary to meet specific nutritional requirements or requirements of a particular product application. For example, the amount of aloe vera extract in a single serving may be tailored to achieve a recommended dose of about five (5) ml to about two-hundred-and-forty (240) ml per day. Also, instead of liquid aloe vera, the semi-solid (i.e., gel) and solid (i.e., powder) forms of aloe vera may be used.

Aloe Vera Processing Methods

Aloe is less commonly consumed in its raw, whole leaf state. Aloe vera processing is complex and varied. However, it is desirable to make the aloe vera gel or juice effective and safe for topical application as well as internal consumption.

Aloe's potential use often involves some type of processing depending on the usage requirements of the end product. Ideally, effective processing methods should be used for the retention of bioactive components imperative for Aloe's therapeutic use.

Preferred methods of processing are those that preserve and maintain almost all the naturally bioactive constituents in the aloe vera leaf. Doing so relies upon processing methodologies like cold-process, whole leaf process, active aloe process, desiccant air dehydration, total process, and a process known as the Time Temperature and Sanitation (TSS) process.

The general processing steps of aloe vera leaves include harvesting and cleaning, hand and mechanical processing, filtration/purification, and stabilization. Methods of aloe vera leaf processing include traditional hand filleting, whole leaf processing, and total process aloe vera.

Traditional hand filleting involves extracting the inner mucilaginous gel from the whole leaf rind without mixing in the bitter aloin from the latex portion. Hand filleting the leaf is the only method to cleanly separate the gel from the rind. The gel is then ground or pulverized into a liquid and the pulp is removed. This hand process, although labor intensive, is one method that removes higher amounts of the anthraquinone laxatives. The rind portion has pigments and the anthraquinones or latex portion of the gel both has a yellow color and is bitter, so removing these portions result in a substantially clear, colorless, flavorless, and odorless aloe vera liquid product.

Whole leaf processing involves dissolving the cellulose (skin/rind) and removing measurable amounts of aloin during processing of the final juice product. This total process is done by a cold process treatment that results in a polysaccharide-rich product. The whole leaf is first pulverized to a soup and then precipitated with chemicals, such as ethanol, carbohydrates, or enzymes (e.g., catalase enzyme, fermentation enzymes in water). These steps decompose the fillet's hexagonal structure to release its constituents. The resulting mash is then mesh-filtered or juice-pressed to remove rind particulates.

The expressed juice passes through a series of filtering columns to remove the unwanted anthraquinone laxatives. The final liquid is held in stainless steel sanitized holding tanks for the de-pulping extractor to remove all residual pulp and to separate it from the juice. The juice is then passed through a series of filters that remove the aloin and aloe-emodin anthraquinones as well as any microscopic particulates. The filtration step uses a carbon-coated plated press filter is used which is attached to the storage tank containing the pre-filtered aloe liquid. This continual filtering process is repeated until ninety-nine percent (99%) of the aloin and aloe-emodin are removed. This filtered product is then placed in a second holding tank with an attached press filter that filters the contents to less than five (5) microns. In other methods, the contents are filtered to below one-half (0.5) micron. This filtering process is repeated until no residue remains. Prior to liquid stabilization, cold filtration processing or distillation is performed for the final purification step. This process produces a constituent-rich juice that is virtually free of bitter laxative anthraquinones.

In one embodiment of the nutritional composition, the plant food is at least 99% free of aloin and aloe-emodin. In another embodiment, the nutritional composition is substantially free of aloe latex. In another embodiment, the plant food is substantially free of solids having a diameter greater than five microns. In yet another embodiment, the plant is substantially free of solids having a diameter greater than one-half micron.

Total-process aloe vera processing is an approach that involves traditional hand filleting of the aloe vera leaves. The leaves are combined with the green rinds and the mucilage layer to produce a product having an increased concentration of valuable constituents, yet one that is virtually free of the anthraquinone purgatives. This process meets the International Aloe Science Council's (IASC) quality standards because it accounts for considerably higher concentrations of aloe's beneficial constituents like calcium, magnesium, malic acid and other total solids.

Other processes include a process known as the Time Temperature Sanitation (TTS) process, desiccant dehydration process, Qmatrix process (a low temperature technology), low temperature short time heat treatment process, and Active Aloe Process which are discussed in detail in C. T. Ramachandra and P. Srinivasa Rao, Processing of Aloe Vera Leaf Gel: A Review, American Journal of Agricultural and Biological Sciences 3 (2), 502-10 (2008).

Present processing techniques aim at producing the best quality aloe product with the greatest quantity of active ingredients. Some of the advantages of whole leaf cold processing methods include maximizing the yield of desirable constituents; cost-effective processing, increased total solids concentration, increased polysaccharide concentration, virtual absence of undesirable anthraquinones, improved desirable characteristics for cosmetic usage, increased concentration of growth factors responsible for accelerating of healing, and increased concentration of factors responsible for the stimulation for the immune system.

In another filtration step, the liquid obtained from processing is filtered to separate the aloin or latex from the liquid, along with the microscopic remains of sand, leaves and other such particles. The filtration process uses a press filter that has various carbon-coated plates. The plates can absorb the latex from the aloe vera liquid. This process is repeated until the whole liquid is free from almost 99% of aloin. As an example, Aloecorp, Inc.'s Aloe Vera Qmatrix 200× powder is made with advanced filtration technology that removes aloin to below 1 part per million (2,000 times lower than the IASC aloin specification standard for certification). After passing through carbon-coated plates, the liquid passes several times through a press filter that contains 5 micron filter paper to completely remove the latex. Finally, the aloe vera liquid is purified using the cold filtration process.

Stabilizing aloe vera liquid or gel that is obtained after processing is important to prevent oxidation of the liquid, which can render the aloe vera product ineffective and unusable. Stabilizing aloe vera gel or liquid can be achieved using cold processing as well as heat treatment processing. Cold processing is done by using enzymes like catalase and glucose oxidase added to the aloe vera gel. These enzymes stop the production of aerobic organisms. Another cold processing method is to expose the aloe vera gel to ultraviolet rays. High-temperature pasteurization of the aloe vera liquid, with the addition of preservatives and additives is also used to stabilize the aloe vera product. Natural preservatives and stabilizers include acetic acid, citric acid, malic acid, alcohol, sugar, honey, glycerin, salt, colloidal silver, ascorbic acid, and vitamin E. Antioxidants such as astaxanthin, grape seed extract, and rosemary extract may also be added to aloe vera.

Distillation technology can alternately be used to separate and remove undesirable constituent fractions from the liquid as well as to stabilize the product without the necessity of additives and preservatives.

Aloe raw materials can be identity graded for purity and quality assurance according to their respective product applications. Four basic grades of raw materials used in finished products are pharmaceutical/medicinal grade, which meets pharmaceutical standards; food grade, which meets standards set for human consumption; cosmetic grade, which meets standards set for cosmetics; and, feed grade, which meets standards set for animal consumption. The difference between each grade type is one of quality and purity. Several criteria are used to judge pharmaceutical grade products. Pharmaceutical grade aloe vera must be in excess of 99% purity with no binders, fillers, excipients, dyes, or unknown substances.

Sea Vegetables

Sea vegetables (also known as algae) include green, brown, red, and blue-green algae. Sea vegetables include kelp (laminaria), which is a brown marine plant that contains vitamins A, B, E, D and K, is a key source of vitamin C, and is rich in minerals. Kelp proteins are comparable in quality to animal proteins. Kelp also contains sodium alginate (algin), an element that helps remove radioactive particles and heavy metals from the body.

Derivatives of algae include chlorophyll, carrageenan, carotenoids (e.g., fucoxanthin), fucoidan (a carbohydrate from brown algae), DNA, and RNA. Algin, carrageenan, and agar are kelp gels that rejuvenate gastrointestinal health and aid digestion. Kelp helps purify blood, relieves arthritis stiffness, and promotes adrenal, pituitary, and thyroid health. Kelp contains iodine, which can be used to normalize thyroid-related disorders.

Kombu (laminaria digitata, setchelli, horsetail kelp) is another sea vegetable that has a long tradition as a Japanese delicacy with great nutritional healing value. It is a decongestant for excess mucous and helps normalize blood pressure. Kombu has abundant iodine, carotenes, B, C, D and E vitamins, minerals like calcium, magnesium, potassium, silica, iron and zinc, and the powerful skin healing nutrient germanium. Kombu is meaty, high-protein seaweed. It is higher in natural mineral salts than most other seaweeds.

Hijiki is a sea vegetable that is a mineral-rich, high-fiber seaweed, with 20% protein, vitamin A, carotenes and calcium. With 1400 mg per 100 g. of dry weight, hijiki has the most calcium of any sea green.

Another sea vegetable, nori (porphyra, laver), is a red sea plant with a sweet, meaty taste when dried. Nori contains nearly 50% balanced, assimilable protein, which is more than any other sea plant. Nori is rich in all the carotenes, calcium, iodine, iron, and phosphorus.

Another sea vegetable, arame (Eisenia bycyclis), is one of the ocean's richest sources of iodine. Herbalists use arame to help reduce breast and uterine fibroids, adhesions, and to normalize menopausal symptoms through arame's fat-soluble vitamins and phytohormones. Arame promotes soft, wrinkle-free skin, enhances glossy hair and prevents its loss. Sea palm (Postelsia Palmaeformis), which is also known as American arame, grows only on the west coast of North America.

Another sea vegetable, bladderwrack, is rich in vitamin K and potassium and is an excellent adrenal stimulant. It is still used by Native Americans in steam baths for arthritis, gout and illness recovery.

Another sea vegetable, wakame (alaria, undaria), is a high-protein, high-calcium seaweed with carotenes, iron, and vitamin C. Wakame is widely used in the Orient for hair growth and luster, and for improved skin tone.

Another sea vegetable, dulse (palmaria palmata), is a red sea plant that is rich in iron, protein, and vitamin A. Dulse has 300 times more iodine and 50 times more iron than wheat. Dulse has purifying and tonic effects on the body, yet its natural, balanced salts nourish as a mineral without inducing thirst.

Another sea vegetable, Irish moss (chondrus crispus, carrageenan), contains electrolyte minerals, calcium, magnesium, sodium, and potassium. Its mucilaginous compounds help the body detoxify, boost metabolism, and strengthen hair, skin and nails.

Sea vegetables or plants are extracted from desiccated or fresh plant material by one or more methods. These methods include decocting, macerating, percolating, tincturing, and distilling. The dry plant material is pulverized into a coarse powder while fresh plant material is chopped into small pieces in order to make a tincture.

A prepared menstruum is a solvent or a combination of solvents such as water, alcohol, wine, vinegar, glycerin, or oil used to extract compounds from plant matter. The menstruum is poured over the plant material until the entire herb is covered. Typically five to six mm of menstruum sits atop the herb; however, if the herb is floating, then the menstruum sits five to six mm below the plant, thereby allowing the plant material to absorb the menstruum.

The menstruum and plant mixture is stirred well so that all the plant material is wet. The vessel is tightly sealed from air. The tincture is agitated frequently for 14 days while the infusion macerates and extracts the phytoactive components. The liquid is decanted, the wet pulp is pressed and the two liquids are combined and filtered, if desired. Various custom mass to volume menstruums can be made by determining the total volume of menstruum need for the quantity of herb on hand and determining the “correct” (a subjective determination) combination and proportion of solvents with the menstruum.

For example, to determine the total volume of menstruum required to make a 1:5 (20%) mass-to-volume tincture having 400 grams of powered dried herb, the weight of the herb is multiplied by 5 (400×5=2000). Therefore, a volume of 2000 ml (2 L) of menstruum is added to 400 grams of herb for 1:5 tincture strength. In addition to liquid extractions, sea vegetables can be used in their solid or powered state and can be combined with various liquid, semi-solid, and solid forms and combinations of aloe vera, humic substances, and sea minerals. Edible and beneficial bacteria may be combined with one or more of the plant and mineral substances such as aloe vera, sea vegetables, shilajit, fulvic acid, humic acid, and sea minerals in their various forms.

In one embodiment, the composition contains about one half (0.5) to about five (5) ml of a 1:5 tincture strength of at least one green, red, brown, blue-green sea plant or algae. Preferably, the composition is filtered to remove all or most of the discernible particulates.

Edible and Beneficial Fungi.

Fungi include edible fungi, yeast foods, beneficial molds, and beneficial yeasts. Edible fungi are simple plant foods that cannot utilize the sun's energy to synthesize their food because they contain no chlorophyll. Types of dietary fungi include caterpillar fungus, Chinese black tree fungus, mushrooms, polyporus umbellate, white jelly fungus, and yeast foods. Fungi have a wealth of health benefits to the body's many systems. Mushrooms comprise the largest group of edible fungi with approximately 38,000 fungi. Edible mushrooms include agaricus blazei murill, enoki, false morel, field, karawatake, lion's mane, maitake, matsutake, nameko, oyster, reishi, shiitake, silver ear, straw, and white. Most mushrooms are specifically beneficial to digestion and immunity. Tremella uniforms is a white jelly fungus that is used against some forms of cancer and may lower total serum cholesterol by preventing absorption of dietary cholesterol.

Yeast foods are foods derived from edible, living, single-celled fungi from various species and strains of saccharomyces cerevisiae that feed on sugar to produce alcohol and carbon dioxide. Types of yeast foods include baker's yeast, brewer's yeast, torula yeast, and vegemite. Beneficial fungi are a group of beneficial microorganisms which types include beneficial molds, beneficial yeasts, as well as the previously mentioned caterpillar fungus.

Beneficial molds are a group of fungi microorganisms that exert beneficial effects on human health. They are characterized by the presence of threadlike filaments (hyphae) that mass together to form mycella (vegetative bodies that resemble cotton). Types of beneficial molds include aspergillus oryzae and rhizopus oligosporus.

Beneficial yeasts are a group of yeasts that are unicellular fungi from the genus saccharomyces. Types of beneficial yeasts include brewer's yeast, saccharomyces boulardii, and torula yeast. Edible and beneficial fungi are commercially available in powder and liquid forms as are their constituent derivatives such as the immune-enhancing polysaccharides like beta-glucans.

Beneficial Bacteria.

Although beneficial bacteria are not usually considered plant foods, beneficial bacteria are included in plant foods for purposes of the present invention. Beneficial bacteria are a group of primitive microorganisms that lack a distinct nuclear membrane and have a cell wall of unique composition. They comprise approximately 90% of the bacteria in a healthy person's digestive tract. Beneficial bacteria have biological functions and provide health benefits to the digestive, excretory, and immune systems in particular. They also enhance the function and production of carbohydrates, lipids, peroxides, and vitamins. Intestinal bacteria also manufacture vitamins. It is widely postulated that a combination of beneficial bacteria works best to adequately replete the colon with a healthy bacterial balance.

Beneficial bacteria are commonly referred to as probiotics and are commercially available in capsule, tablet, powder, and liquid forms. Groups of beneficial bacteria include bifidobacteria, enterococcus faecium, lactobacillus, propionibacterium freudenreichi, and streptococcus.

In one embodiment, the composition contains up to 750 mg of one or more (complex) edible and beneficial fungi and bacteria in a powdered form for standard gel encapsulation. Edible and beneficial fungi and bacteria may be combined with one or more of aloe vera, shilajit, fulvic acid, humic acid, sea vegetables, and sea minerals in their various forms.

Mineral Foods

Humic Substances.

Shilajit (or humate) is a mineral food formed through the biological and geological processes of natural materials, is a black to brown (tarry) exudate obtained from rocks that are commonly found in the mountains of the Himalayas. Shilajit may also be obtained from the rocks in mountain regions of Asia, Europe, North America, and South America and even in the Antarctic continent. Although it is obtained from rock (mineral sources), Shilajit also contains plant-sourced matter (from fossilized plants).

Shilajit contains two soluble fractions of organic plant acids that may be extracted from humus found in soil, sediment, or aquatic environments. Of these organic plant acids, humic acids are the major extractable component extracted with alkaline extraction from humate. Humic acids appear dark-brown to black in color. Another organic plant acid, fulvic acid, is the bio-stimulating fraction extracted from humate and typically appears yellow or gold to orange or yellow-brown in color. The terms “humic substances” and “humates” refer collectively to the acid radicals found in humic matter that are typically separated from humic matter by alkaline extraction. Humates are in essence the salts of humic acid and fulvic acid.

Shilajit commonly contains (in milligrams per 100 grams)

| | |
|---|---|
| Carbohydrates | 70,430 |
| Lipids | 90 |
| Minerals | 12,320 |
| Calcium | 1,200 |

Barium

Magnesium

Phosphorus

Iron

Zinc

Manganese

Potassium

Chromium

Cobalt

Copper

Silicon

Sodium

Strontium

Sulphur

Titanium

Organic Acids:

Fulvic Acid

Humic Acid

| | |
|---|---|
| Benzoic Acid | 8,580 |
| Hippuric Acid | 6,130 |
| Proteins: | 9,250 |
| Water: | 7,910 |

Proteins: 9,250

Water: 7,910

Other: Urea, Ichthyol, Dibenzo-alpha-pyrones (DBP)

Humic acid (molecular formula C₉H₉NO₆) is the acid radical found in humic matter that is soluble in alkalis and weak acids. Humic acid is insoluble in strong aqueous acids (pH<2), methyl ethyl ketone, and methyl alcohol. Humic acid molecules form complexes having an average chemical formula of C₁₃₇H₁₃₆O₈₉N₉.

Fulvic acid (molecular formula C₁₄H₃₂O₈) is an acid radical portion of humic substances that is soluble in water at any pH, in methyl ethyl ketone, and in methyl alcohol. Fulvic acid is a weak organic acid with a pH of about 4. In one embodiment, 12% fulvic acid solution has a pH between 1.8-2.5, specific gravity of 1.01-1.03, an electrical conductivity of 3800-5550 mS/cm, and a concentration of 3.5%-5.5%. Preferably, fulvic acid has a pH of 1.98, a specific gravity of 1.014, an electrical conductivity of 5227 mS/cm, and a concentration of 4.65%.

The structure of fulvic acid is best characterized as a loose assembly of aromatic organic polymers with many carboxyl groups (COOH) that release hydrogen ions, resulting in species that have electric charges at various sites on the ion. In contrast to its molecular formula, fulvic acid complexes (i.e. an organic polymer) have an average chemical formula of C₁₃₅H₁₈₂O₉₅N₅S₂. A hydrogen-to-carbon ratio greater than 1:1 indicates less aromatic character (i.e., fewer benzene rings in the structure), while an oxygen-to-carbon ratio greater than 0.5:1 indicates more acidic character than in other organic fractions of humus (e.g., humic acid, the other natural acidic organic polymer that can be extracted from humus).

Fulvic acid is believed to originate as a product of microbial metabolism, although it is not synthesized as a life-sustaining carbon or energy source. Fulvic acid is especially reactive with metals and forms strong complexes with Fe3+, Al3+, and Cu2+.

In contrast to humic acid, fulvic acid is able to penetrate the cell and mitochondria of a cell because of its smaller molecular size. Fulvic acid has higher oxygen content, but lower carbon content than humic acid. Compared to humic acid, fulvic acid also contains more acidic functional groups, particularly COOH groups. The total acidity of fulvic acid (typically between 900-1400 meq/100 g) is considerably higher than the acidity of humic acid (typically 400-870 meq/100 g).

Fulvic acid commonly contains amino acids, minerals, and electrolytes as major components. Fulvic acid mineral complex contains several amino acids, including alanine, glutamic acid, glycine, histidine, isoleucine, leucine, methionine, phenylalanine, serine, threonine, tryptophan, and valine. In addition to these amino acids, fulvic acid contains over 77 minerals, trace minerals, and electrolytes.

Attributes of Fulvic Acid

Powerful Organic Electrolyte.

Fulvic acid is an organic natural electrolyte that can balance and energize biological processes. Fulvic acid has proven to be a powerful organic electrolyte, serving to balance cell life. Fulvic acid has the outstanding ability to accomplish this objective in numerous ways.

Promotes Electrochemical Balance.

Electrochemical balance is required for health. Fulvic acid is a powerful natural electrolyte. Fulvic acid is available at times as an electron donor and at other times as an electron acceptor, based on the cell's requirements for balance. One reaction that occurs is an oxidation reaction, in which the chemical species loses electrons as a donor. Another reaction is a reduction in which the active species gains electrons as an acceptor. Trace minerals in fulvic acid mineral complexes, described above, also serve as electrodes to conduct current in vivo.

Powerful Natural Free Radical Scavenger and Antioxidant.

Fulvic acid free radical scavengers behave as electron donors or acceptors, depending upon the need for balance in a given situation. Fulvic acid can take part in oxidation-reduction reactions with transition metals. In one embodiment of the invention, an aqueous solution has a negative oxidation-reduction potential.

Complexes and Dissolves Minerals and Trace Elements.

Due to its polyfunctionality, humic substances are one of the most powerful chelating agents among natural organic substances. The zwitterionic character of humic substances allows the interaction of anions with positively charged groups of humics and cations with negative charged groups of these substances. Fulvic acid is especially active in dissolving minerals and metals when in solution with water and converting these minerals into new minerals. The metallic minerals simply dissolve into ionic form, and disappear into the fulvic structure becoming biochemically reactive and mobile. Fulvic acid transforms these minerals and metals into fulvic acid complexes that have vastly different characteristics from their metallic mineral form. Fulvic acid naturally chelates metallic minerals, turning them into readily absorbable bioavailable forms. Fulvic acid also has the unique ability to weather and dissolve silica. Dietary fulvic acids assist the body in maintaining proper mineral balance by its ability to chelate excess minerals, as well as heavy metals, and assist in supplying the body a safe, natural organic plant form of minerals in the proper balance nature intended.

Enhances Nutrients

Fulvic acid enhances the availability of nutrients and makes them more readily absorbable.

It also allows minerals to regenerate and prolongs the residence time of essential nutrients. It prepares nutrients to be metabolized by cells. It allows nutrients to with one another, breaking them down into the simplest ionic forms to be chelated by the fulvic acid electrolyte.

Transports Nutrients.

Fulvic acid readily complexes with minerals and metals making them easily absorbable through cell walls. Fulvic acids also dissolve and transport vitamins, coenzymes, auxins, hormones, and natural antibiotics that are generally found throughout the soil, making them available. These substances are effective in stimulating even more vigorous and healthy growth. These substances are produced by certain bacteria, fungi, and actinomycetes in decomposing vegetation in the soil. It has been determined that all known vitamins can be present in healthy soil. Plants manufacture many of their own vitamins, yet these from the soil further supplement the plant. Upon ingestion, these nutrients are easily absorbed by animals and humans in their natural plant form. Fulvic acid can often transport many times its weight in dissolved minerals and elements.

Catalyzes Enzyme Reactions.

Fulvic acid has close association with enzymes, increasing their activity. It increases activity of enzymes, and especially influences respiratory catalysts. Fulvic acids increase the activity of several enzymes essential to healthy assimilation including alkaline phosphatase, transaminase, and invertase.

Increases Assimilation.

Fulvic acid metal organic complexes are of a low molecular weight and therefore have a small molecular size. Their small size allows for a high degree of penetration into cells. Fulvic acid complexes and chelates are able to readily pass through semi-permeable membranes such as cell walls. Humate substances like humic and fulvic acid contain many healing phytochemicals and enzymes which readily disperse throughout the body, even to the cell's interior cytoplasm and nucleus.

Stimulates Metabolism.

Plant cells exposed to fulvic acid exhibit improved growth and fulvic acid appears to cause the genetic mechanism of plants to function at a higher level. The observed improvements in plant growth attributed to fulvic acid can similarly benefit in humans and animals. For example, plant cells exposed to fulvic acid exhibit improved growth because fulvic acid aids in penetrating roots to quickly transport nutrients to the shoots of plants. Also, oxygen is absorbed more intensely in the presence of fulvic acids, so fulvic acid can help relieve oxygen deficiency and increase the vital activity of cells. Also, fulvic acids change the pattern of the metabolism of carbohydrates, resulting in an accumulation of soluble sugars. These soluble sugars in crease the pressure of osmosis inside the cell wall and enable plants to withstand wilting. Further, fulvic acid enhances growth and may stimulate the immune system.

Detoxifies Pollutants

An important aspect of humic substances is related to their absorptive interaction with environmental chemicals, either before or after they reach concentrations toxic to living organisms. The toxic herbicide known as Paraquat is rapidly detoxified by humic substances (fulvic acids). Fulvic acids have a special function with respect to the demise of organic compounds applied to soil as pesticides. Fulvic acid is vital in helping to form new species of metal ions, binding with organic pollutants such as pesticides and herbicides, and catalyzing the breakdown of toxic pollutants. Radioactive substances react rapidly with fulvic acid, and only a brief time is required for equilibrium to be reached. All radioactive elements are capable of reacting with fulvic acid and thus forming organometallic complexes of different adsorptive stability and solubility. Fulvic acids are especially important because of their ability to form complexes of, or chelate, metal ions and interact with silica. They have the ability to bio-react with one another and also to inter-react with cells to synthesize or even transmute new mineral compounds. Fulvic acids act as specific cell sensitizing agents and enhance the cell membrane's permeability. They stimulate and balance cells to create optimum growth and replication conditions.

Dissolves Silica.

Interactions between fulvic acid and metal ions or silica may increase their aqueous concentrations to levels above that of natural dissociation alone.

Synthesizes or Transmutates Minerals.

Fulvic acid complexes have the ability to bio-react one with another and also inter-react with cells to synthesize or transmutate new mineral compounds. The transmutation of vegetal silica and magnesium to form calcium in animal and human bones is an example of new synthesis of minerals.

Enhances Cell Division and Elongation.

Fulvic acid stimulates and balances cells, creating optimum growth and replication conditions.

Enhances the Permeability of Cell Membranes.

Fulvic acids act as specific cell sensitizing agents and enhance the permeability of the cell membrane.

Increases Metabolism of Proteins.

Fulvic acid intensifies the metabolism of proteins, RNA, and DNA, It has been found that fulvic acid increases DNA content in cells and also enhances RNA synthesis rates.

Catalyzes Vitamins within the Cell.

Fulvic acid has the ability to complex vitamins into its structure, where they are presented to the cell in combination with complex minerals. In this natural condition, nutrients are catalyzed and utilized by the cell.

Chelates all Monovalent and Divalent Elements.

Fulvic acid has the power to form stable water-soluble complexes with monovalent, divalent, trivalent, and polyvalent metal ions. It can aid the actual movement of metal ions that are normally difficult to mobilize or transport. Fulvic acids are excellent natural chelators and cation exchangers, and are important to cell nutrition.

Provides Missing Link in the Food Chain.

Fulvic acid contains natural macro- and micro-nutrients, phytochemicals, anti-viral and antibiotic agents that both inhibit and destroy disease-causing pathogens while strengthening and regulating the immune system.

In one embodiment, the nutritional composition contains about fifteen (15) to about thirty (30) ml of food-grade 100% fulvic acid liquid mineral complex solution. The fulvic acid solution is clear, virtually colorless (a pale amber), odorless, and tasteless in non-chlorinated water. The fulvic acid solution is preferably free of preservatives, additives, and heavy metal contaminants. Optionally, the fulvic acid solution is medicinal grade. In another embodiment, a humic-fulvic acid mineral complex liquid or powder is used.

Minerals

General forms of minerals include inorganic salts, organic acid salts, and amino acid chelates. Inorganic salts are minerals bonded with phosphates, sulfates, chlorides and are important for electrolyte balance. Organic acid salts are minerals bonded with ascorbates, acetates, and citrates. Organic acid salts are natural and more effectively absorbed by the digestive system than inorganic salts. Amino acid chelates bind to amino acids for improved mineral absorption. Specific minerals chelate best with specific amino acids. Organic minerals, or mineral foods, are available as a powder form or as a liquid suspension.

Zeolites are minerals that have a porous structure and are available in a powder form or as a liquid suspension. Zeolites are hydrated alumino-silicate minerals with an “open” structure that can accommodate a wide variety of positive ions such as sodium, potassium, calcium, and magnesium. Clinoptilolite is one zeolite used for therapeutic purposes.

There are more than 150 types of Zeolites have been synthesized and 48 types of naturally-occurring Zeolites are presently known including amicite, analcime, barrerite, bellbergite, bitkitaite, boggsite, brewsterite, chabazite, clinoptilolite, cowlesite, dachiardite, edingtonite, epistibite, erionite, faujasite, ferrierite, garronite, gismondine, gmelinite, gobbinsite, gonnardite, coosecreekite, harmotome, herschelite, heulandite, laumontite, levyne, maricopaite, mazzite, merlinoite, mesolite, montesommaite, mordenite, natrolite, offretite, paranatrolite, paulingite, phillipsite, pollucite, scolecite, sodium dachiardite, stellerite, tetranotrolite, thomsonite, tschernichite, wairakite, wellsite, willhendersonite, and yugawaralite.

Bentonite clay is a colloidal aluminum silicate. Montomorillonite clay is a type of colloidal clay that is often used for therapeutic use. Charcoal (also known as active charcoal) is a porous, solid product obtained when carbonaceous materials such as cellulose or wood are partially burned in the absence of air. Charcoal is almost 100% carbon. Charcoal is often described as a rigid sponge because its enormous surface area provides for great absorption ability. Diatomaceous earth: fossilized remains of microscopic shells created by diatoms (one-celled plants). Its principal constituent is silicon dioxide. Dolomite: is a substance that is derived from ground Limestone.

Salts derived from the earth or the ocean may be used as a food seasoning, food preservative, and as a mineral supplement. Salts include sodium chloride, unrefined sea salts, and sea minerals.

Sea minerals include sea salt and deep sea minerals. Sea salts are salts derived from oceans and some inland water bodies after evaporation of water. Sea salt generally contains magnesium, calcium, other minerals, and trace elements. More specifically, sea salt has approximately 84 trace+minerals. Ions commonly included in unrefined sea salt include (by mass percent) chloride (Cl⁻, 55.03%; sodium (Na⁺), 30.59%; sulfate (SO₄²⁻), 7.68%; magnesium (Mg²⁺), 3.68%; calcium (Ca²⁺), 1.18%; potassium (K⁺), 1.11%; bicarbonate (HCO₃⁻), 0.41%; bromide (Br⁻), 0.19%; borate (BO₃³⁻), 0.08%; strontium (Sr²⁺), 0.04%; and other ions, 0.01%. Although the salinity of sea water varies worldwide, the relative amounts of the constituent ions remain the same.

Sea salt generally contains magnesium, calcium, and other minerals and trace elements. Unrefined sea salt, such as French sea salt, is produced from sea water that has been pooled into basins and the water evaporated. Because it is unrefined, it contains a higher mineral and trace mineral content than refined sea salt. Sea salt from geothermal caves is known to have more than 80 mineral elements. Sea salt allows liquids to freely cross body membranes, e.g., the glomeruli of the kidney or blood vessel walls. Sea salt is necessary for the proper breakdown of plant carbohydrates into usable and assimilable nutrients.

Deep sea minerals are a mixture of expired plankton, diatoms and other minute sea life from 2,000 feet below the sea and have been shown to be highly assimilable in studies comparing blood mineral content prior and post digestion. Deep sea minerals are naturally ionized because they have been part of a living organism. Therefore, deep sea minerals are easily assimilable through digestive systems and through cell walls. Deep sea minerals are bioprocessed minerals that contain approximately 68 nutritionally-necessary trace elements in the most bioavailable form. The mineral and electrolyte balance of deep sea minerals mimics the composition of human blood serum and has approximately the same pH of human blood (7.35-7.45). Sea minerals, such as deep sea minerals, are preferably free of heavy metals such as arsenic, cadmium, mercury, lead, radon, ruthenium, and uranium, as well as high levels of non-biological mineral such as aluminum, titanium, etc.

Deep sea minerals are also highly assimilable due to unique incorporation within single celled phytoplankton and diatoms. The concentration of electrolytes in deep sea minerals exceeds that of other mineral sources. Sea minerals are available in crystal or powder form that can be dissolved into solution to create a saturated solution. For example, a saturated aqueous solution contains 26% sea salt.

In one embodiment, the composition contains about one (1) to about five (5) ml of twenty-six percent (26%) saturated solution of deep sea minerals that are free of heavy metals and contaminants. The saturation is preferably made with pure, non-chlorinated water. The resulting solution substantially resembles water in clarity, color odor and taste. That is, the solution is substantially clear, colorless, and odorless, and has a taste similar to that of spring water.

Combined Bioactivity of Aloe and Fulvic Acid

Aloe vera (or aloe) and fulvic acid combine to form a composition that exceeds its singular bioactivity because of its combined synergistic action. The combination of aloe and fulvic acid offers a complete compliment of macro- and micro-nutrient components with improved bioavailability. The combination also provides improved absorption of these components.

Aloe and fulvic acid combine to provide a more expansive and extensive array, availability, and concentration of highly bioactive macro- and micro-nutrients in a single source. Fulvic acid can readily dissolve in water and complex with aloe's many minerals, amino acids, vitamins, enzymes, hormones, and other natural agents making them more biochemically reactive and mobile.

Fulvic acid can transport many times its weight of these dissolved minerals and elements to cells to effectively stimulate cellular metabolism, energy, and growth. Fulvic acid metal organic complexes have a low molecular weight, and because of this, they have a small molecular size, making them capable of a high degree of penetration into cells. Fulvic acid also enhances the availability of aloe's nutrients, making them more readily absorbable, and prolonging the nutrients' residence time in the cell, the place where nutrients are most needed.

Aloe improves the solubility of water-soluble and lipid-soluble compounds. Aloe can hydrate cell membranes to allow fulvic acid materials to penetrate cell walls. Additionally, aloe's nutrients can chelate with fulvic acid and together are transported into the cell more efficiently by aloe's penetrative action.

The biological activity of aloe can add to and even synergize with many agents, such as those in fulvic acid, to produce therapeutic effects. In combination, fulvic acid and aloe offer a greater alkalizing potential on the body than when consumed independently. At about 17′C, pure aloe vera juice has a pH of 7.5; in comparison, fulvic acid isolate has a pH of 2.8. Mixing equal quantities of aloe vera juice and fulvic acid isolate results in a pH of 4.2. Fulvic acid and aloe also share antibiotic, antibacterial, antiviral, antifungal, and anti-inflammatory agents. When combined, the activity of these agents improves due to the synergistic effects of aloe and fulvic acid acting together.

The combination of aloe and fulvic acid improves protein synthesis and utilization. Fulvic acid provides amino acids—the building block of proteins. When aloe and fulvic acid combine, they share amino acids and enzymes. The presence of aloe's complete amino acid profile (22 in all), combined with the amino acids in fulvic acid, results in the potential to amplify a number of other yet-to-be-identified components. For example, the combination of aloe and fulvic acid very likely results in a composition having twenty to thirty additional enzymes that are not present in either aloe or fulvic acid alone.

By their very nature, enzymes are highly interactive proteins and often assume personalities of one another. By serving as electrodes, trace minerals in the fulvic acid electrolyte benefit protein synthesis. Because aloe enables the bowel to absorb nutrients more efficiently, especially proteins, the combination of aloe and fulvic acid improves protein synthesis and absorption.

In one embodiment, the nutritional composition has between one and about 240 ml of pure-grade fermented- or carbohydrate-derived 100% liquid fulvic acid isolate added to one-half (0.5) liter of non-chlorinated water. The fulvic acid can be processed to meet purity and quality standards via micron filtration between 0.5 and 5 microns. The resulting solution substantially resembles water in clarity, color, odor, and taste. That is, the solution is substantially clear, colorless, and odorless, and has a taste similar to that of spring water. Also, the solution is free of preservatives, additives, heavy metals, aloe latex, anthraquinones, and atoin.

The invention is not limited to naturally-occurring macro- and micro-nutrients. The composition may contain organic, inorganic, and/or synthesized macro- and micro-nutrients. Additionally, the composition may be combined with bacteria, viruses, molds/fungi, nutraceutical, bioceutical, cosmeceutical, dermaceutical, pharmaceutical, smart drug, and/or nanoceuticals for delivery to cells.

To make various compositions of the present invention, ingredients in any of their liquid, gel, and/or solid forms may be mixed together to produce a liquid, semi-solid (e.g., gel or paste), or a solid (e.g., powder) product. In some formulations, ingredients are mixed with water to produce a beverage, preferably a beverage that resembles water in that it is substantially clear, substantially colorless, substantially odorless, and/or substantially flavorless.

One embodiment of the present invention is an aqueous solution containing aloe vera distillate and fulvic acid isolate. To about 0.5 liter of purified drinking water, between about one ml and about 300 ml of aloe vera distillate and about one ml to about 240 ml of fulvic acid isolate may be added. The resulting mixture is substantially clear, colorless, flavorless, and odorless.

In another embodiment, the nutritional composition contains aloe vera distillate and fulvic acid isolate. To achieve a mixture that is substantially clear, colorless, flavorless, and odorless, the ratio of aloe vera to fulvic acid is between about 1:3 and 4:1. In some embodiments, the ratio of aloe vera to fulvic is about 2:1, 1.5:1, or 1:1. For example, 45 ml of aloe vera distillate are mixed with 30 ml of fulvic acid isolate, resulting in a substantially clear, colorless, odorless, and flavorless liquid.

Example Formulations.

The following are examples of some usable formulations.

Formula 1.

To one liter of purified drinking water, add 20 ml 100% fractionally-distilled whole leaf aloe vera liquid and 15 ml carbohydrate-derived fulvic acid liquid (fulvic isolate). Preferably, the aloe vera liquid is from aloe vera barbadensis miller that has been fractionally distilled to remove at least 99% of aloe-emodin and aloin. One acceptable aloe vera liquid is George's 100% aloe vera that is fractionally distilled from aloe vera leaves and made by Warren Labs in Abbott, Tex. One acceptable fulvic acid isolate is Wu Jin San Fulvic Acid Supplement (concentrated fulvic acid in pure rainwater from the sea) made by Bio-Agricultural Services, Inc. in Independence, Oreg.

Formula 2.

To 0.5 liters of non-chlorinated drinking water, add between 1 and about 300 ml of 100% aloe vera liquid extract and about 1 to about 240 ml 100% fulvic acid liquid mineral complex solution. Add about 1 ml of 26% saturated aqueous sea salt solution. The amounts of aloe vera liquid extract and fulvic acid mineral complex may be adjusted to eliminate a discernible flavor, color, or odor in the resulting solution.

Formula 3.

Combine 250 mg aloe vera whole leaf powder with 250 mg shilajit/humate powder and mix. Instead of the shilajit/humate powder, 20 ml of humate/fulvic complex solution or fulvic acid isolate (or equivalent powder or extract) may be used. As an option, add 250 mg of powdered plant food (or equivalent extract or solution) containing sea vegetables, fungi, and/or beneficial bacteria.

Formula 4.

Combine 45 ml of aloe vera inner leaf or whole leaf juice (100%) with 20 ml of humic/fulvic complex solution (or shilajit/humate powder) and 1 ml of 26% saturated aqueous mineral food solution containing sea minerals, zeolite, etc. Fulvic acid isolate liquid may be used in place of the humic/fulvic complex solution. As an option, add 1 ml of plant food extract (or equivalent solution or powder) containing sea vegetables, fungi, beneficial bacteria, etc.

The composition may be combined with other nutrients in their organic or inorganic/synthesized states to provide a delivery system that assists in carrying biological components to cells for improved efficacy. The composition delivers not only the biological components of aloe vera to cells, but also other macro- and micro-nutrients, herbs, supplements, and substances with which it combines or interacts. Nutrient sources include plant food, mineral food, their derivatives and synthetic analogs/mimetics and combinations thereof. For example, ATP is an endogenous compound that contains adenine, ribose, and three phosphate groups (inorganic forms of phosphorus). Other substances that may combine with the composition of the present invention include alcohols, alkaloids, auxins (such as cytokinins, indoles, and kinetin), carbohydrates, proteins, amino acids, enzymes, hormones, caroteinoids (including carotenes and xanthophylls), glycosides, lignans, lipids, microorganisms, organic or inorganic minerals, zeolites, nucleic compounds (including constituents of nucleic acids and bases, nucleosides, nucleosomes, and nucleotides), pharmaceutical drugs and “smart” drugs, phenolic compounds, quinones, vitamins, gases (including oxygen, hydrogen peroxide, ozone, carbon dioxide), herbs and foods, genetically modified organisms, microorganisms, nucleic compounds (including nucleic acids and the various bases that are constituents of the molecules of nucleic acids, nucleosides, nucleosomes, and nucleotides), organic and inorganic acids, cosmeceuticals, bioceuticals, dermaceuticals, nanoceuticals, quantum substances, nanoparticles, and lyposomes. These substances preferably combine with the plant food or the mineral food in a ratio of up to 5 parts plant food (e.g., aloe vera) or mineral food (e.g., fulvic acid) to 1 part of the substance to be added (i.e., a 5:1 ratio). This ratio may be up to 10:1 or higher, depending on the efficacy of the particular substance (e.g., 50:1, 100:1, 500:1) and may also be up to 1:10.

Smart drugs enhance mental or physical performance and include synthetic compounds designed to improve the chemical structure of nutrients that exist in nature and provide concentrated forms of naturally-occurring substances.

In quantum-altered substances, mechanical, chemical, electrical, optical, quantum, or other process or combination thereof alters the physical or chemical characteristics of the substance to create a new form or species of the substance. For water-containing substances, quantum alteration creates a new form or species of water which, when utilized by plants, animals or humans, demonstrates measurable and repeatable benefits to chemical, enzymatic and general cellular functions.

Lyposomes, also known as niosomes, are artificial vesicles or sacs (similar to an air bubble) comprised of any of the phospholipids that constitute cell membranes. Liposomes mimic the chemical structure of cell membranes. Liposomes can fuse with cells and thereby deliver their internal load. The inner space of liposomes can be loaded with any other drugs/nutrients (i.e. active ingredients) and used as delivery systems to deliver these active-ingredients to their target sites. This aspect of liposomes also applies to topically-delivered compounds. Forms of liposomes include alpha hydroxy acid liposomes, anti-irritant liposomes, ethosomes (firming, humectant, and moisturizing) liposomes, PEGylated liposomes, sunscreen liposomes, nutrient liposomes. Various types of liposomes include ethosomes, and PEGylated liposomes (liposomes subjected to process of PEGylation.)

Mycelization is a relatively new process which breaks up fat soluble vitamins/nutrients into small particles so they become water soluble. Like liposomes, the mycelization process substantially increases the body's absorption of these nutrients and increases their incorporation into cell membranes.

Nutraceutical originated from the terms “nutrition” and “pharmaceutical”. A nutraceutical is a food, or part of a food, that provides medical or health benefits, including the prevention and/or treatment of a disease. However, the term nutraceutical has no regulatory definition. Nutraceuticals are commonly referred to as functional food/beverage, including herbals and dietary supplements.

Bioceuticals are plant-based substances with known health benefits. Essentially they are premium-grade natural formulas, remedies, and dietary supplements derived from plants and their derivatives and used as natural alternative to pharmaceutical drug therapy in treating common health concerns.

Cosmeceuticals are substances and products that are applied externally (topically) to the hair, skin, and teeth for appearance enhancement purposes, including products that are applied topically in order to derive specific therapeutic benefits. Cosmeceuticals include cosmetics, personal care, hygiene, body care, skin protectors, skin improvers, hair and scalp improvers, oral/dental care and oral/dental hygiene, toiletries, and other beauty and appearance enhancement agents. Cosmeceuticals are cosmetic grade finished products.

Dermaceuticals are defined as natural ingredient-based topical face and body products designed to restore the natural ability of collagen-based tissue, such as hair, skin, and nails, to maintain its health. Similar to cosmeceuticals, dermaceuticals employ high strength natural ingredients like foods, mineral, and botanicals over chemical-based preparations found in cosmeceuticals. More currently they may also include oral products, such as beverages with antioxidants that are designed to improve the physical outward appearance of aging from the inside out. Dermaceuticals are generally cosmetic-grade finished products if used externally, but can be food grade if used internally.

Nanoceuticals are nano-manufactured nanoscale substances that can be used in the making of a pharmaceutical, bioceutical, nutraceutical, cosmeceutical, and dermaceutical. Nanoceuticals have properties of material with dimensions at the nanoscale which range roughly from 1 to 100 nanometeres (nm), or more simply put, they are nanomaterials at the atomic and molecular level. Nanoscale materials can have different properties. Nanoscale materials also have far larger surface areas than similar volumes of larger-scale materials, meaning that more surface is available for interactions with other materials in proximity. For nano-sized nutrients, this theoretically increases the availability of the nutrient to the medium. The other primary advantage of nanomaterials like nano-sized drugs, nutrients and nano-sized versions of foods, food ingredients, and dietary supplements is that their tiny size allows them access to tiny places in the body, making them more readily available.

Some common nanotechnology applications and products are drug-delivery techniques, water-filtration techniques, nanofilms (such as anti-microbial films), nanotubes, nanoscale transistors, and solar plastics. Specifically in drug-delivery techniques, dendrimers are a type of nanostructure that can be precisely designed and manufactured for a wide variety of applications, including treatment of cancer and other diseases. Dendrimers carrying different materials on their branches can do several things at one time, such a recognizing diseased cells, diagnosing disease states (including cell death), drug delivery, reporting location, and reporting outcomes of therapy.

Importantly, nanoscale objects behave quite differently from those at larger scales.

Therefore, properly structuring and combining nanoscale particles can make for new and interesting applications and products. Currently, nanotechnology is being used to filter and purify water, a process that may have nano engineering implications on the extracting, filtering, and purification of other natural substrates such as botanicals like aloe and minerals like humate. Nanotechnology using controlled-release nanomaterials are nano product technologies, particularly antioxidant and antimicrobial nano products. Nanoprocessing and nanoproduction of foods are useful for nutrient delivery, for improving bioavailability of nutrients, and for providing stability and protection to sensitive nutrients from moisture, oxidation, and the like. Nanonutrition is achieved through self-assembled, naturally occurring liquids comprised of micelles or liposomes, or nanoencapsulates, the tiniest of capsules that form naturally when nature requires a hydrophobic substance like a fat-soluble substance to be soluble in water. These nanoemulsions and nanodispersions, like other colloidal systems, are used in food and beverages. Micelles or liposomes assist in emulsifying or dissolving fat in water solutions (such as fat globules in milk). Nanotechnology can be used to incorporate nano-sized ingredients, such as minerals, into baked goods, cereals, and other food products to improve the nutrient profile.

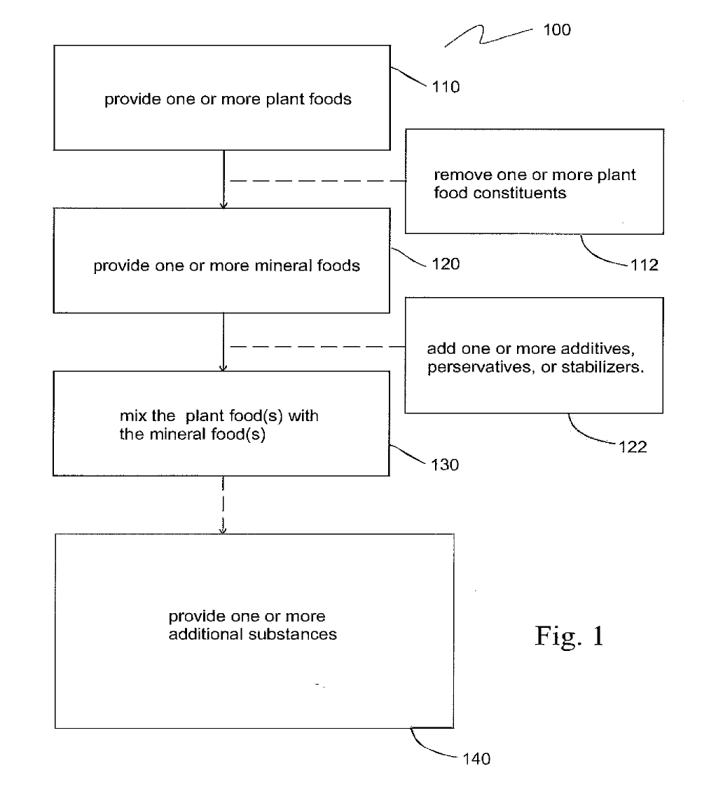

FIG. 1 illustrates various embodiments of a method 100 of making a nutritional composition. The dashed lines extending from steps 112, 122, and 140 represent optional steps in method 100. The steps of method 100 may be performed in the order as shown in FIG. 1 or in any other order as appropriate.

Step 110 is to provide one or more of plant foods. Plant foods include aloe vera, green algae, red algae, brown algae, blue-green algae, kelp, kombu, hijiki, nori, arame, sea palm, bladderwrack, wakame, dulse, carrageenan, fungi, and bacteria. Plant foods may be in solid, semi-solid (gel) or liquid forms. Step 120 is to provide one or more mineral foods. Mineral foods include shilajit, humic acid, fulvic acid, bentonite clay, charcoal, diatomaceous earth, dolomite, fossilized remains, rocks, gems, sodium chloride, sea salt, and derivatives or combinations thereof. Mineral foods may also be in solid, semi-solid (gel), and liquid forms. Step 130 is to mix the plant food(s) with the mineral food(s). Preferably, plant food(s) to mineral food(s) has a ratio of about 1:3 to about 4:1. The mixing step may be performed in water by, for example, creating an aqueous solution of the ingredients.

Step 112, an optional step, is to remove one or more constituents of the plant food. These constituents include aloin, aloe-emodin, anthraquinones, aloe latex, and particles having a diameter greater than 5 microns. Step 122, an optional step is to add one or more preservatives, additives, or stabilizers. These preservatives, additives, and stabilizers include acetic acid, citric acid, ascorbic acid, malic acid, alcohol, honey, glycerin, salt, colloidal silver, potassium sorbate, sodium benzonate, vitamin E, astaxanthin, grape seed extract, and rosemary extract. Step 140, an optional step, is to provide one or more additional substances for combination with the plant food(s) and the mineral food(s). The additional substances include alcohols, alkaloids, auxins, carbohydrates, proteins, amino acids, enzymes, hormones, caroteinoids, glycosides, lignans, lipids, microorganisms, minerals, zeolites, nucleic compounds, pharmaceutical drugs, “smart” drugs, phenolic compounds, quinones, vitamins, gases, herbs, foods, genetically modified organisms, microorganisms, nucleic compounds, organic acids, inorganic acids, cosmeceuticals, bioceuticals, dermaceuticals, nanoceuticals, quantum-altered substances, nanoparticles, and lyposomes.

The features and advantages described herein are not all-inclusive and, in particular, many additional features and advantages will be apparent to one of ordinary skill in the art in view of the specification and claims. Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

I claim:

1. A nutritional composition comprising:
at least one plant food selected from the group consisting of aloe, green algae, red algae, brown algae, blue-green algae, kelp, kombu, hijiki, nori, arame, sea palm, bladderwrack, wakame, dulse, carrageenan, fungi, and bacteria; and
at least one mineral food selected from the group consisting of shilajit, humic acid, fulvic acid, bentonite clay, charcoal, diatomaceous earth, dolomite, fossilized remains, rocks, gems, sodium chloride, sea salt, and derivatives or combinations thereof.

2. The nutritional composition of claim 1, wherein aloe is an extract of aloe barbadensis miller.

3. The nutritional composition of claim 1, wherein the aloe is substantially free of anthraquinones.

4. The nutritional composition of claim 1, wherein the aloe is at least 99% free of aloin and aloe-emodin.

5. The nutritional composition of claim 1, wherein the plant food contains less than 1 ppm aloin.

6. The nutritional composition of claim 1, wherein the plant food is substantially free of solids having a diameter greater than 5 μm.

7. The composition of claim 1, further comprising at least one of acetic acid, citric acid, ascorbic acid, malic acid, alcohol, honey, glycerin, salt, colloidal silver, potassium sorbate, sodium benzoate, vitamin E, astaxanthin, grape seed extract, and rosemary extract.

8. The nutritional composition of claim 1, wherein the composition comprises at least 1000 mg of mucopolysaccharides per liter.

9. The nutritional composition of claim 1, wherein the mineral food is fulvic acid.

10. The nutritional composition of claim 9, wherein the fulvic acid is one of a fermentation-derived fulvic acid liquid and a carbohydrate-derived fulvic acid liquid.

11. The nutritional composition of claim 9, wherein the composition has a volume-to-volume ratio of aloe vera to fulvic acid between about 1:3 to about 4:1.

12. The composition of claim 9, wherein the fulvic acid contains at least 1 part dibezo-alpha-pyrones per 20 parts fulvic acid.

13. The nutritional composition of claim 9, wherein the composition is an aqueous solution.

14. The nutritional composition of claim 13, wherein the aqueous solution has at least one property selected from the group consisting of substantially clear, substantially colorless, substantially odorless, and substantially flavorless.

15. The nutritional composition of claim 9, further comprising a fulvic acid complex of least one of Fe³⁺, Al³⁺, and Cu²⁺.

16. The nutritional composition of claim 9, further comprising a fulvic acid mineral complex.

17. The nutritional composition of claim 1, wherein the composition has a volume-to-volume ratio of the at least one plant food to the at least one mineral food that is between 2:1 and 1:2.

18. The nutritional composition of claim 1, wherein the nutritional composition contains from 20 to 50 grams of at least one of fungi and bacteria per liter of the at least one plant food.

19. The nutritional composition of claim 1 further comprising at least one substance selected from the group consisting of alcohols, alkaloids, auxins, carbohydrates, proteins, amino acids, enzymes, hormones, caroteinoids, glycosides, lignans, lipids, microorganisms, minerals, zeolites, nucleic compounds, pharmaceutical drugs, “smart” drugs, phenolic compounds, quinones, vitamins, gases, herbs, foods, genetically modified organisms, microorganisms, nucleic compounds, organic acids, inorganic acids, cosmeceuticals, bioceuticals, dermaceuticals, nanoceuticals, quantum-altered substances, nanoparticles, and lyposomes, wherein the ratio of the at least one plant food to the at least one substance is 10:1 or less.

20. A method of making a nutritional composition comprising the steps of:
providing at least one plant food at least one plant food selected from the group consisting of aloe, green algae, red algae, brown algae, blue-green algae, kelp, kombu, hijiki, nori, arame, sea palm, bladderwrack, wakame, duise, carrageenan, fungi, and bacteria; and
providing at least one mineral food selected from the group consisting of shilajit, humic acid, fulvic acid, bentonite clay, charcoal, diatomaceous earth, dolomite, fossilized remains, rocks, gems, sodium chloride, sea salt, and derivatives or combinations thereof; and
mixing the at least one plant food with the at least one mineral food, wherein the ratio of the at least one plant food to the at least one mineral food is from about 1:3 to about 4:1.

21. The method of claim 20, wherein the mixing step is performed in water.

22. The method of claim 20, further comprising the step of removing at least one of aloein, aloe-emodin, anthraquinones, aloe latex, particles having a diameter greater than 5 microns.

23. The method of claim 20, further comprising the step of adding one ingredient selected from the group consisting of acetic acid, citric acid, ascorbic acid, malic acid, alcohol, honey, glycerin, salt, colloidal silver, potassium sorbate, sodium benzoate, vitamin E, astaxanthin, grape seed extract, and rosemary extract.

24. The method of claim 20, further comprising the step of adding at least one ingredient selected from the group consisting of alcohols, alkaloids, auxins, carbohydrates, proteins, amino acids, enzymes, hormones, caroteinoids, glycosides, lignans, lipids, microorganisms, minerals, zeolites, nucleic compounds, pharmaceutical drugs, “smart” drugs, phenolic compounds, quinones, vitamins, gases, herbs, foods, genetically modified organisms, microorganisms, nucleic compounds, organic acids, inorganic acids, cosmeceuticals, bioceuticals, dermaceuticals, nanoceuticals, quantum-altered substances, nanoparticles, and lyposomes, wherein the ratio of the at least one plant food to the at least one ingredient is 10:1 or less.
